(19) United States
(12) Patent Application Publication
(10) Pub. No.: US 2024/0105345 A1
(43) Pub. Date: Mar. 28, 2024

(54) AUTOMATIC RANKING AND RANK ORDER DISPLY OF MEDICAL INFORMATION, AND ASSOCIATED DEVICES, SYSTEMS, AND METHODS

(71) Applicant: KONINKLIJKE PHILIPS N.V., Eindhoven (NL)

(72) Inventors: Pieter Christiaan VOS, VUGHT (NL); Ralf Dieter HOFFMANN, BRUEGGEN (DE); Gertjan Laurens SCHUURKAMP, UTRECHT (NL)

(21) Appl. No.: 18/266,899

(22) PCT Filed: Dec. 7, 2021
(86) PCT No.: PCT/EP2021/084585

§ 371 Date: Jun. 13, 2023

Related U.S. Application Data
(60) Provisional application No. 63/127,012, filed on Dec. 17, 2020.

(51) Int. Cl. G16H 50/70; G16H 10/60; G16H 50/20

(52) U.S. Cl. CPC G16H 50/70; G16H 10/60; G16H 50/20

(57) ABSTRACT

A system may include a data store and a processor circuit in communication with the data store and a user input device. The data store may include clinical records associated with patients. For each of the patients, the clinical records may include data corresponding to a set of inputs associated with a medical condition of the patient and data corresponding to a set of outcomes associated with the medical condition of the patient. The processor circuit may be configured to obtain the clinical records via the data store and to receive, via the user input device, a selection of a driving outcome from among the set of outcomes. The processor circuit may be configured to determine a first ranking of the set of inputs based on the driving outcome and a classification model and to provide, at a display, a screen display including a graphical representation of the set of inputs automatically arranged based on the first ranking.

### TECHNICAL FIELD

The present disclosure relates generally to systems for automatically ranking and providing a representation of the ranking of a set of variables associated with clinical records. In particular, the set of variables (e.g., types of patient data) may be ranked based on a selection of a driving outcome, such as a treatment plan, and a classification model, which may be used to determine a relationship between the variables and the outcome.

### BACKGROUND

To make a diagnosis and/or select a treatment for a current patient, a clinician may compare medical data associated with the current patient to medical data associated with a set of other patients. For instance, a clinician may attempt to identify patients that share similar symptoms, age, family history, and/or other medical data with the current patient. By identifying similar patients, the clinician may make a more confident diagnosis and/or may select a more suitable treatment for the current patient. For example, in selecting a treatment, the clinician may predict an outcome of the treatment on the current patient based on the outcome of the treatment on the identified patients.

In some cases, more relevant similarities between different patients may enable a clinician to make a more reliable diagnosis than other, less relevant similarities between patients. To that end, factors that contribute the most to (e.g., act as the greatest predictor of) an outcome, such as a diagnosis, may be more relevant for identifying similar patients than factors that contribute little to the diagnosis. For instance, for a particular diagnosis, a patient's age may be more relevant than the patient's gender in identifying similar patients. However, relevant similarities between patients may be difficult to discern, especially in multidisciplinary fields and with increasing types of heterogeneous medical data derived from different clinical disciplines.

### SUMMARY

Embodiments of the present disclosure are directed to systems, devices, and methods for ranking and providing a representation of the ranking of a set of input variables associated with clinical records. In particular, using clinical records associated with a plurality of patients, the set of input variables (e.g., types of patient data), such as demographics, medical records, family history, test results, and/or the like associated with each patient, may be ranked based on a selection of a driving outcome and a classification model. The driving outcome may correspond to a diagnosis, a treatment plan (e.g., administration of a medicine, performance of a medical procedure, and/or the like), and/or the like. The classification model, such as a random forest classifier, may be used to determine a relationship (e.g., a correlation) between the variables and the outcome. In particular, the classification model may determine a relative importance (e.g., feature importance) of each of the set of input variables on the driving outcome, and the ranking of the set of input variables may be determined based on this relative importance. A graphical representation of the set of input variables automatically arranged based on the ranking may further be provided in a screen display on a display device, such as a monitor. Thus, an indication of the relationship between the set of input variables and the driving outcome may be provided to a user, such a clinician. In addition, the plurality of patients may include a current patient and a set of additional patients. Accordingly, the indication of the relationship between the set of input variables and the driving outcome may enable a user to identify similar patients to the current patient based on relevant similarities (e.g., relevant input variables) to the driving outcome. To that end, the relevance of the similarities between the current patient and other patients may be readily apparent and/or optimized based on the relationship between the set of input variables and the driving outcome. As such, the data associated with the similar patients may be used to make a reliable diagnosis, to select a suitable treatment plan, and/or the like for the current patient.

In an exemplary aspect, a system, includes a data store and a processor circuit. The data store may include clinical records associated with a plurality of patients. For each of the plurality of patients, the clinical records may include data corresponding to a set of inputs associated with a medical condition of the patient and data corresponding to a set of outcomes associated with the medical condition of the patient. The processor circuit is in communication with the data store and a user input device. The processor circuit may configured to: obtain the clinical records via the data store; receive, via the user input device, a selection of a driving outcome from among the set of outcomes; determine a first ranking of the set of inputs based on the driving outcome and a classification model; and provide, at a display in communication with the processor circuit, a screen display including a graphical representation of the set of inputs automatically arranged based on the first ranking.

In some aspects, the processor circuit may be configured to, in response to receiving a selection of a filter criterion associated with the set of inputs via the user input device: determine a second ranking of the set of inputs based on the driving outcome, the filter criterion, and the classification model; and modify the screen display provided on the display to present the graphical representation of the set of inputs automatically arranged based on the second ranking. In some aspects, the filter criterion corresponds to at least one of: a selection of a subset of the clinical records based on data corresponding to an input of the set of inputs, or a selection of a subset of the set of inputs. In some aspects, the processor circuit may be configured to determine the second ranking further based on: identifying, based on the filter criterion, a filtered dataset comprising a subset of the data corresponding to the set of inputs and the data corresponding to the set of outcomes; and identifying, based on the classification model and the filtered dataset, a respective correlation between each input of the set of inputs associated with the filtered dataset and the data corresponding to the driving outcome associated with the filtered dataset. In some aspects, the screen display comprises a set of icons. Each of the set of icons correspond to a respective input of the set of inputs. In some aspects, the processor circuit may be configured to provide the graphical representation of the set of inputs automatically arranged based on the first ranking based on a first arrangement of the set of icons, and the processor circuit may be configured to present the graphical representation of the set of inputs automatically arranged based on the second ranking based on a second arrangement of the set of icons.

In some aspects, the screen display may include a graphical representation of the set of outcomes. In some aspects, the processor circuit may be further configured to: output, to the screen display, the graphical representation of the set of outcomes based on the data corresponding to the set of outcomes; and in response to receiving a selection of a filter criterion associated with the set of inputs via the user input device: identify a filtered dataset, which may include a subset of the data corresponding to the set of outcomes; and modify the graphical representation of the set of outcomes based on the subset of the data corresponding to the set of outcomes.

In some aspects, the processor circuit may be configured to determine the first ranking further based on: identifying, based on the classification model, a respective correlation between the data corresponding to each input of the set of inputs and the data corresponding to the driving outcome. In some aspects, the classification model comprises at least one of a random forest, a logistic regression model, or a cox regression model. In some aspects, the set of inputs may include at least one of an age, gender, race, symptom, clinical test result, family history, or diagnosis. In some aspects, the set of outcomes may include at least one of a treatment, a mortality rate, a morbidity rate, a level of severity, or a diagnosis confidence metric associated with the medical condition.

In some aspects, the processor circuit may be further configured to: provide, at the display, a graphical representation of the data corresponding to the set of inputs based on the graphical representation of the set of inputs. In some aspects, the processor circuit may be further configured to: in response to receiving a selection of a filter criterion associated with the set of inputs via the user input device: identify a filtered dataset, wherein the filtered dataset comprises a subset of the clinical records based on data corresponding to an input of the set of inputs; and modify the graphical representation of the set of inputs based on the subset of the clinical records. In some aspects, the graphical representation of the data corresponding to the set of inputs comprises a histogram.

In an exemplary aspect, a method may include obtaining, by a processor circuit, clinical records associated with a plurality of patients via a data store. For each of the plurality of patients, the clinical records may include data corresponding to a set of inputs associated with a medical condition of the patient and data corresponding to a set of outcomes associated with the medical condition. The method may further include: receiving, at the processor circuit, a selection of a driving outcome from among the set of outcomes via a user input device; determining, by the processor circuit, a first ranking of the set of inputs based on the driving outcome and a classification model; and providing, by the processor circuit, a screen display including a graphical representation of the set of inputs automatically arranged based on the first ranking on a display in communication with the processor circuit.

In some aspects, the method may include: in response to receiving, at the processor circuit, a selection of a filter criterion associated with the set of inputs via the user input device: determining, by the processor circuit, a second ranking of the set of inputs based on the driving outcome, the filter criterion, and the classification model; and modifying, by the processor circuit, the screen display provided on the display to present the graphical representation of the set of inputs automatically arranged based on the second ranking. In some aspects, the method may include: identifying a treatment for a patient of the plurality of patients based on the graphical representation of the set of inputs arranged based on the first ranking; and performing the treatment.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of diagnostic analysis system, according to aspects of the present disclosure.

FIG. 2 is a schematic diagram of a processor circuit, according to aspects of the present disclosure.

FIG. 3 is a schematic diagram of a data store, according to aspects of the present disclosure.

FIG. 4 is a flow diagram of a method of ranking a set of input variables associated with clinical records based on a particular outcome associated with the clinical records, according to aspects of the present disclosure.

FIG. 5 is a diagrammatic view of a screen display associated with clinical records, according to aspects of the present disclosure.

FIG. 6 is a diagrammatic view of a screen display with a graphical representation of input variables associated with clinical records arranged based on a ranking, according to aspects of the present disclosure.

FIG. 7 is a diagrammatic view of a screen display with a graphical representation of filter criteria associated with clinical records, according to aspects of the present disclosure.

FIG. 8 is a diagrammatic view of a screen display with a graphical representation of input variables associated with clinical records arranged based on a ranking, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

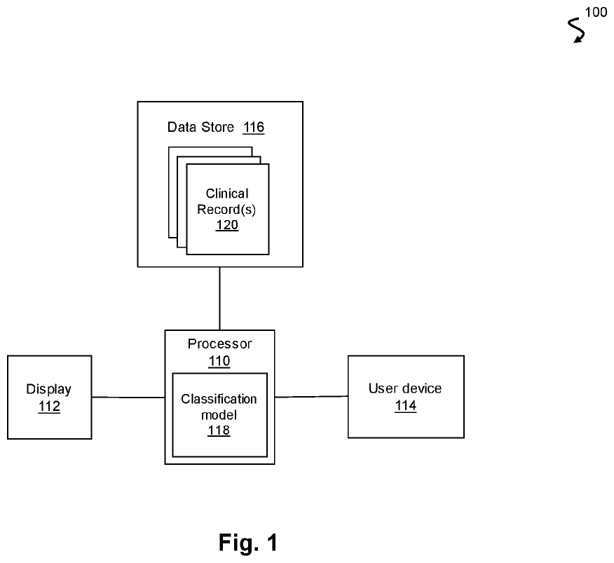

FIG. 1 is a schematic diagram of a diagnostic analysis system 100, according to aspects of the present disclosure. The diagnostic analysis system 100 includes a processor 110 in communication with a display 112, a user device 114 (e.g., a user input device), and a data store 116. The processor 110 may be configured to implement a classification model 118, such as a random forest (e.g., a random decision forest), and the data store 116 may include clinical records 120, which may include information associated with a plurality of patients. As described herein, the diagnostic analysis system 100 may be used to provide a comparison between clinical records associated with different patients. In particular, the diagnostic analysis system 100 may support a clinician in selecting an optimal treatment for a particular patient based on a comparison of clinical records associated with other, similar patients.

The processor 110 may also be described as a processor circuit, which can include other components in communication with the processor 110, such as a memory, a communication interface, and/or other suitable components. The processor 110 may include a central processing unit (CPU), a graphical processing unit (GPU), a digital signal processor (DSP), an application specific integrated circuit (ASIC), a controller, a field programmable gate array (FPGA) device, another hardware device, a firmware device, or any combination thereof configured to perform the operations described herein. The processor 110 may also be implemented as a combination of computing devices, e.g., a combination of a DSP and a microprocessor, a plurality of microprocessors, one or more microprocessors in conjunction with a DSP core, or any other such configuration.

The data store 116 may also be described as a database, memory, or storage. The data store 116 may be any suitable storage device, such as a cache memory (e.g., a cache memory of the processor 134), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, solid state drives, other forms of volatile and non-volatile memory, or a combination of different types of memory. Additionally or alternatively, the data store 116 may be implemented on a server, or cloud server. To that end, the data store 116 may be accessed directly (e.g., locally) or remotely by the processor 110.

The data store 116 can be configured to store the clinical records 120 relating to a patient's medical history, history of procedures performed, anatomical or biological features, characteristics, or medical conditions associated with a patient. In particular, the clinical records 120 may include data corresponding to inputs (e.g., input variables), such as demographics (e.g., age, gender, ethnicity, a behavior, and/or the like), medical history, family history, symptoms, clinical test results (e.g., blood work, images, a genomic test result, etc.), and/or the like, associated with a patient and/or a medical condition of the patient, and the clinical records 120 may include data corresponding to outcomes, such as treatment options, condition severity, confidence metrics, and/or the like associated with the patient and/or a medical condition associated with the patient. The clinical records 120 may include other forms of medical history, such as but not limited to images, videos, and/or any imaging information relating to the patient's anatomy. The data store 116 can also be configured to store computer readable instructions, such as code, software, or other application, as well as any other suitable information or data.

The user device 114 may be an input/output (I/O) device. For instance, the user device 114 may include a mouse, a keyboard, a button, a scroll wheel, a joystick, a microphone, and/or the like configured to receive an input from a user. The user device 114 may also include a display, such as display 112, a speaker, a light, and/or the like configured to provide an output to the user. Further, the user device 114 may be a user computing device, such as a phone, tablet, laptop, computer, and/or the like. Moreover, in some embodiments, the user device 114 may be communicatively coupled to the processor 110 so that a user input received at the user device 114 is communicated to the processor 110.

In some embodiments, components of the diagnostic analysis system 100 may be communicatively coupled via any suitable communication link (e.g., a wireless or a wired connection). For example, a combination of the processor 110, display 112, user device 114, or the data store may be coupled via a wired link, such as a universal serial bus (USB) link or an Ethernet link. Alternatively, the processor 110, display 112, user device 114, or the data store be wirelessly coupled, such as via an ultra-wideband (UWB) link, an Institute of Electrical and Electronics Engineers (IEEE) 802.11 WiFi link, or a Bluetooth link.

The processor 110 may be configured to process the clinical records 120. For instance, the processor 110 may be configured to employ the classification model 118 to rank input variables (e.g., to perform feature ranking) associated with the clinical records 120. In particular, the processor 110 may use the classification model 118 to rank the input variables based on one or more of the outcomes associated with the clinical records 120, such as a selected outcome (e.g., a driving outcome). For instance, the classification model 118 may rank the input variables based on a relationship (e.g., a correlation) between each of the input variables and the selected outcome. The classification model 118 may be a random forest. Additionally or alternatively, any suitable classifier, such as a support vector, regression classifier (e.g., a linear regression model or a logistic regression model), a cox regression model, and/or the like may be employed as the classification model 118. In particular, a classification model configured to perform feature ranking and/or to determine a p-value and/or an odds ratio (e.g., a measure of association) based on the input variables, such that the input variables may be ranked, may be employed as the classification model 118.

In some embodiments, the processor 110 may receive a user input via the user device 114. For instance, the processor 110 may receive selection of a subset of the input variables, a selection of a filter criterion associated with the clinical records, a selection of the driving outcome, and/or the like. Accordingly, the processor 110 may determine the ranking of the input variables described above further based on the user input.

The display 112 is coupled to the processor 110. The display 112 may be a monitor or any suitable display (e.g., electronic display). The processor 110 may be configured to output a screen display including a graphical representation of the input variables arranged according to the ranking to the display 112. For instance, the processor 110 may arrange icons or other suitable graphical representations of the set of input variables in an order determined based on the ranking. As an illustrative example, the processor 110 may arrange an icon corresponding to an input variable with the greatest impact (e.g., highest correlation) on a driving outcome first within the screen display and may arrange an icon corresponding to an input variable with the least impact (e.g., lowest correlation) on the driving outcome last within the screen display. As such, a clinician may rapidly identify relationships between the various input variables and the driving outcome. Thus, the clinician may more readily determine a diagnosis and/or a treatment plan for a particular patient, as described in greater detail below.

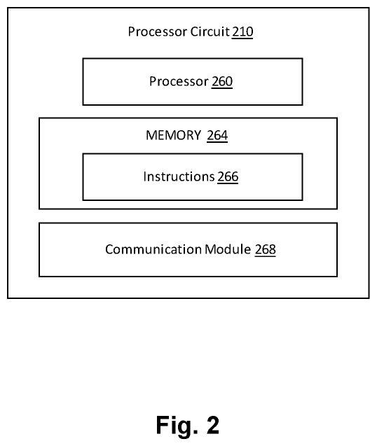

FIG. 2 is a schematic diagram of a processor circuit 210, according to embodiments of the present disclosure. The processor 110 of FIG. 1 may be implemented as the processor circuit 210. In an example, the processor circuit 210 may be in communication with the display 112, the user device 114, and/or the data store 116. One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 260, cause the processor 260 to perform the operations described herein with reference to the processor 110 (FIG. 1). Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 210, the user device 114, the display 112, and/or the data store 116. In that regard, the communication module 268 can be an input/output (I/O) device. In some instances, the communication module 268 facilitates direct or indirect communication between various elements of the processor circuit 210 and/or the processor 110 (FIG. 1).

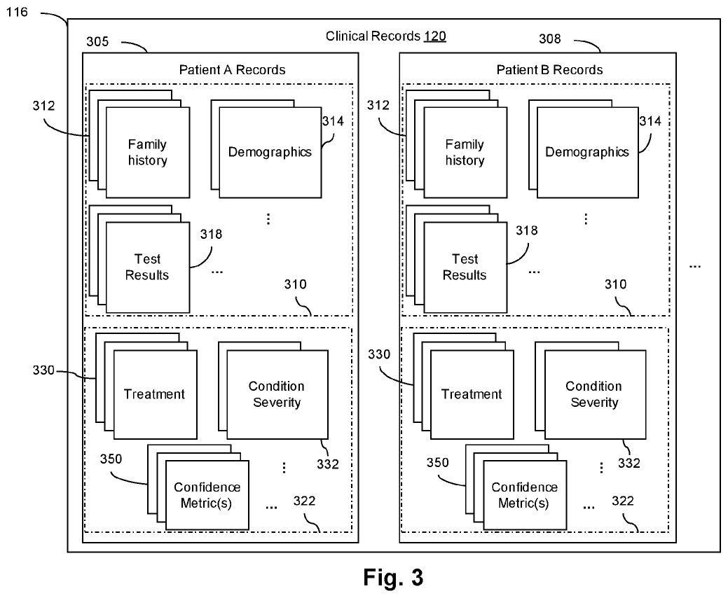

As shown in FIG. 3, a plurality of clinical records 120 may be stored in the data store 116. For example, a first patient record 305 associated with a first patient (e.g., “Patient A”) and a second patient record 308 associated with a different, second patient (e.g., “Patient B”) are depicted in FIG. 3. A patient record (e.g., 305, 308) may contain any suitable types of data or files corresponding to a patient's health, history, or anatomy, such as electronic health records or electronic medical records. As illustrated, for example, each patient record (305, 308) may include data corresponding to a set of input variables 310 (e.g., a set of inputs), as well as data corresponding to a set of outcomes 322. The input variables 310 may include such family history 312, which may correspond information related to genetic disorders, demographics 314 (e.g., age, gender, race, ethnicity, and/or the like of a patient), test results 318 (e.g., clinical test results), such as images, biopsy results, blood work, and/or the like, among other variables. The input variables 310 may additionally or alternatively include symptoms experienced by a patient, procedures performed on a patient, or any other suitable medical record. To that end, the input variables 310 may characterize a patient with respect to a medical condition (e.g., a diagnosis) and/or a medical history. The outcomes 322 may include a treatment 330 and/or a treatment plan, a condition severity 332, such as a cancer stage in the case of a cancer diagnosis, an estimated morbidity (e.g., morbidity rate), a mortality rate, a recurrence rate, a time to recurrence and/or the like, and/or a confidence metric 350. The confidence metric 350 may be associated with a diagnosis, a likelihood of success with respect to a treatment, and/or the like. The outcomes 322 illustrated and described herein are intended to be exemplary and not limiting.

In some cases, patients may have varying levels of data in a clinical record. For instance, the first patient record 305 may include a result corresponding to a particular diagnostic test within the test results 318, while the second patient record 308 may lack a result corresponding to the diagnostic test. In some cases, the data corresponding to the test results 318 of the second patient record 308 may be populated with a default (e.g., predetermined) and/or null value to indicate the absence of the result. Additionally or alternatively, some input variables 310 and/or outcomes 322 may only be relevant to a particular population of patients. As such, the data values for those input variables 310 and/or outcomes 322 may be populated with a default and/or null value for patients excluded from the population.

As further shown in FIG. 2, clinical records 120 may include many more records than the two depicted (e.g., 305, 308). For instance, clinical records 120 may include one, two, five, ten, 100, 1000, and/or any suitable number of clinical records. Similarly, additional input variables 310 and/or outcomes 322 than the illustrated quantities may be included in each clinical record of the clinical records 120.

As previously mentioned, the data store 116 may be any suitable storage device, or a combination of different types of memory. For example, the first patient record 305 may be stored on one storage device, including any type of storage device previously listed, and the second patient record 308 may be stored on a separate storage device. The first storage device may be in communication with the second storage device, and the two may subsequently be in communication with the processor 110 of FIG. 1. Similarly, the total number of clinical records 120 may be stored on any number of storage devices in communication with one another and with the processor 110. In addition, all or some of the clinical records 120 may be stored on a server, or cloud server, and accessed remotely by the processor 110. All or some of the clinical records 120 may further be copied such that a second copy or back-up of all or some of the clinical records 120 are stored on a separate storage device, server, or cloud-based server.

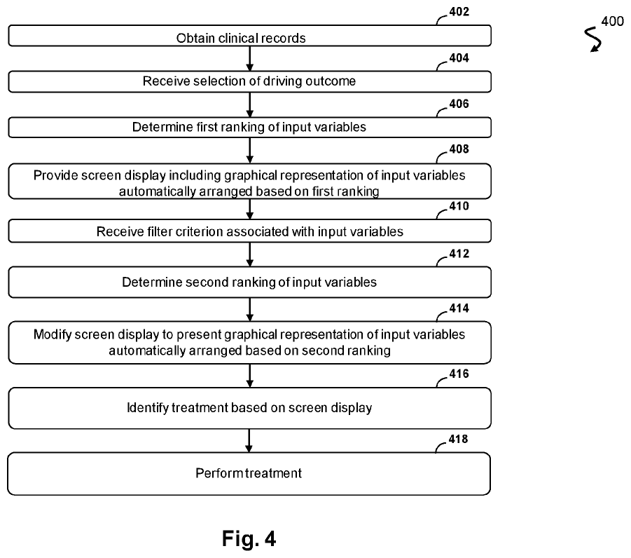

FIG. 4 is a flow diagram of a method 400 of ranking a set of input variables associated with clinical records based on a particular (e.g., a driving) outcome associated with the clinical records, according to aspects of the present disclosure. One or more steps of the method 400 can be performed by a processor circuit of the diagnostic analysis system 100, including, e.g., the processor 110 (FIG. 1). As illustrated, method 400 includes a number of enumerated steps, but embodiments of method 400 may include additional steps before, after, or in between the enumerated steps. In some embodiments, one or more of the enumerated steps may be omitted, performed in a different order, or performed concurrently.

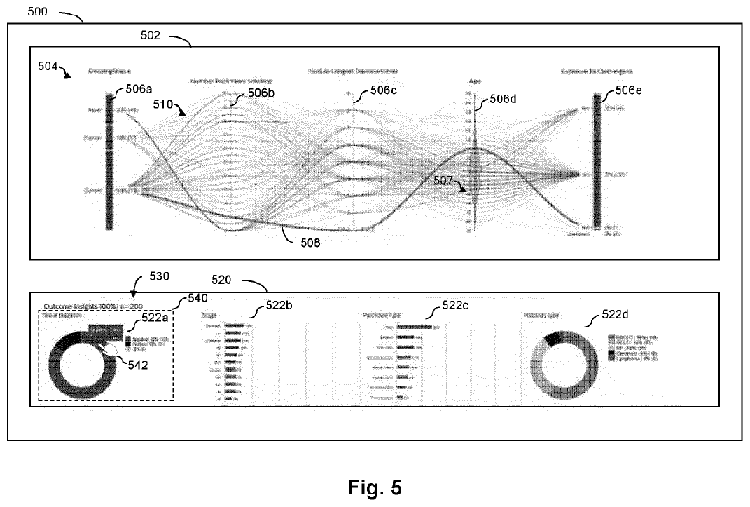

The method 400 may further be used to provide an indication (e.g., a graphical representation) of the ranking of the set of input variables at, for example, a display. Accordingly, one or more steps of the method 400 will be described with reference to FIGS. 5-8, which provide schematic diagrams of a screen display (e.g., graphical user interface (GUI)), according to aspects of the present disclosure. For the purposes of example, FIGS. 5-8 depict graphical representations associated with the diagnosis and treatment of lung cancer. However, embodiments are not limited thereto. Instead, it may be appreciated that the techniques described herein may be applied to any suitable clinical application.

At step 402, method 400 includes obtaining clinical records. In particular, clinical records, such as clinical records 120, may be obtained from a data store (e.g., data store 116). As described with reference to FIG. 3, the clinical records may be associated with a plurality of patients. Moreover, for each patient, the clinical records may include data corresponding to input variables (e.g., inputs), such as input variables 310, as well as data corresponding to outcomes (e.g., outcomes 322).

In some embodiments, step 402 may involve accessing a remote data store, such as a cloud-based server, or a local data store, such as a local memory. As an illustrative example, the processor 110 of FIG. 3 may transmit a request for the clinical records 120 from the data store 116, and in response to the request, the data store 116 may transmit the clinical records 120 to the processor 110.

Further, in some embodiments, a screen display may be provided based on the obtained clinical records. For instance, the processor 110 may output the screen display to the display 112. FIG. 5 provides a diagrammatic view of an exemplary screen display 500 (e.g., graphical user interface (GUI)) that may be provided based on the obtained clinical records. As shown in FIG. 5, the screen display 500 may include an input window 502 (e.g., a first area or region of the screen display 500) associated with the input variables of the clinical records and an outcome window 520 (e.g., a second area or region of the screen display 500) associated with the outcomes of the clinical records. The screen display 500 may also provide a graphical representation of a sample size 530. The sample size 530 may indicate the total number and/or the percentage of clinical records used to determine the information displayed with respect to the input window 502 and the output window 520.

The input window 502 may include a parallel plot 504 of the data corresponding to the input variables associated with the clinical records. In this way, the input window 502 simultaneously displays the values of data associated with each of a first input variable 506a (e.g., smoking status), a second input variable 506b (e.g., number pack years smoking), a third input variable 506c (e.g., nodule longest diameter (mm)), a fourth input variable 506d (e.g., age), and a sixth input variable 506e (e.g., exposure to carcinogens) for a set of patients. The parallel plot 504 may include respective graphical representations of the input variables 506a-e based on the type of data corresponding to the input variables 506a-e. For instance, the illustrated first input variable 506a and the sixth input variable 506e are shown with graphical representations that reflect qualitative data, such as a patient's response to a questionnaire, as well as a distribution of patients within the different categories of the qualitative data (e.g., in terms of a percentage of total patients and a raw number of patients belonging to each category). Further, the illustrated second input variable 506b, third input variable 506c, and fourth input variable 506d are shown with graphical representations that reflect quantitative data. Moreover, the fourth input variable (e.g., age) is illustrated with a histogram 507, which shows the distribution of patients (e.g., clinical records corresponding to patients) across a spectrum of ages. In particular, the vertical axis of the histogram 507 corresponds to patient ages, and the horizontal axis of the histogram 507 corresponds to the quantity of patients whose data (e.g., within a clinical record) indicates that the patient is a particular age. The illustrated graphical representations of the input variables 506a-e are intended to be exemplary and not limiting. To that end, any suitable graphical representation (e.g., graph, chart, histogram, icon, symbol, text, and/or the like) may be used to represent the input variables 506a-e.

As further illustrated, the parallel plot 504 may identify the data (e.g., the clinical record) corresponding to a particular patient, such as a current patient whose clinical record is under evaluation by a clinician. That is, for example, the parallel plot 504 may plot a current patient curve 508 alongside a set of similar patient curves 510 to provide a comparison between the current patient and other patients. The current patient curve 508 may be distinguishable based on a style (e.g., line thickness, dots, dashes, color, highlighting), labeling, and/or other difference between the current patient curve 508 and the similar patient curves 510.

The output window 520 may include a graphical representation of the outcomes associated with the clinical records. For instance, the illustrated output window 520 includes a graphical representation of a first outcome 522a (e.g., tissue diagnosis), a second outcome 522b (e.g., stage), a third outcome 522c (e.g., procedure type), and a fourth outcome 522d (e.g., histology type).

It should be appreciated that the graphical representations illustrated in FIG. 5 and described herein are intended to be illustrative and not limiting. To that end, an input variable associated with the clinical records may be represented in any suitable manner, such as with text, a symbol, an icon, a chart, a plot, and/or the like. Similarly, an outcome associated with the clinical records may be represented in any suitable manner, such as with text, a symbol, an icon, a chart, a plot, and/or the like.

In the illustrated embodiment of the screen display 500, a relationship (e.g., a correlation) between any of the displayed input variables 506a-e and the outcomes 522a-d is not readily apparent. As an illustrative example, based on the screen display 500, it is unclear whether a patient's smoking status (506a) or the number of pack years smoking (506b) associated with the patient is a better predictor of (e.g., is more strongly correlated with) a particular tissue diagnosis (522a), such as a positive tissue diagnosis. To that end, it may be difficult to determine which input variables to use to identify patients similar to (e.g., to group or cluster with) the current patient. A treatment or diagnosis determined based on patients identified as similar to the current patient based on more relevant input variables for a particular outcome, may be more reliable than a treatment or diagnosis determined based on patients identified as similar to the current patient based on less relevant input variables for the outcome. Accordingly, by providing an indication of a ranking of the importance (e.g., impact) of input variables with respect to an outcome, such as the importance in predicting the outcome, patient care may be improved.

At step 404, method 400 includes receiving a selection of a driving outcome. The driving outcome may be an outcome selected from among a set of outcomes (e.g., outcomes 322) that may be used to determine a ranking of the input variables associated with the clinical records, as described in greater detail below. In some embodiments, the selection of the driving outcome may be received via a user device (e.g., user device 114). For instance, the selection may correspond to a user input received via the user device. To that end, the selection may involve a user clicking, highlighting, circling, typing the name of the driving outcome, selecting the driving outcome from a list (e.g., a dropdown) and/or the like. In some embodiments, for example, the selection may correspond to a user interaction with a screen display (e.g., screen display 500 of FIG. 5).

For instance, as shown in FIG. 5, the selection of the driving outcome may correspond to a cursor 542 interaction (e.g., click) with a particular outcome (e.g., the first outcome 522a). In some cases, in response to the selection of an outcome as the driving outcome, the screen display 500 may be updated to provide a graphical representation of the driving outcome. For example, the selected driving outcome may be outlined, highlighted, may change color, size, and/or style, and/or the like. Additionally or alternatively, the outcomes that were not selected as the driving outcome may be hidden from the outcome window 520.

At step 406, the method 400 includes determining a first ranking of the input variables. In particular, the first ranking of the input variables may be determined based on the selected driving outcome and a classification model, such as the classification model 118. For instance, the diagnostic analysis system 100 may determine, based on the classification model, a respective correlation between the data corresponding to each input variable of the set of input variables and the data corresponding to the driving outcome. As described above, the classification model may be a random forest classifier, a support vector, regression classifier (e.g., a linear regression model or a logistic regression model), a cox regression model, and/or any suitable classifier. For the purposes of example, portions of the method 400 are described herein with reference to a random forest classifier as a classification model. However, embodiments are not limited thereto. To that end, any suitable classification model configured to perform feature ranking and/or to determine a p-value and/or an odds ratio (e.g., a measure of association) based on the input variables, such that the input variables may be ranked, may be employed to perform one or more portions of the method 400. For instance, a classification model may determine the first ranking of the input variables based on identifying the input variables that have the greatest effect on the driving outcome according to a feature ranking, a p-value, and/or an odds ratio.

A random forest classifier is an ensemble algorithm that combines a multitude of trained decision trees, which may vary slightly from one another, via random sampling. The random forest classifier may output the mode of the classes (e.g., outcomes) determined during the classification and associated with the decision trees. The random forest classifier may be used for supervised classification, where an output (e.g., a classification) is known in advance. During training of a random forest classifier, the random forest classifier may provide an internal ranking of a feature importance (e.g., a feature ranking). The internal ranking may identify the features (e.g., the input variables) that have the greatest effect on the output (e.g., the outcomes), as well as the features that have the least effect on the output (e.g., the outcomes). As an illustrative example, a number between 0 and 10 may be assigned to each feature input to the random forest, where 0 represents the lowest effect on an output and 10 represents the greatest effect on the output. Moreover, the internal ranking may be performed in relatively short time (e.g., in real-time), such as on the order of milliseconds. By training the random forest classifier using the clinical records (e.g., clinical records 120), the random forest classifier may automatically provide a feature ranking representative of the correlation between the data corresponding to each input variable of the set of input variables and the data corresponding to the driving outcome. In particular, the data corresponding to the set of input variables may be provided as inputs to the random forest classifier and may be mapped to a known outcome (e.g., according to supervised classification) according to the data corresponding to the outcomes. The internal feature ranking of the random forest classifier may then be used to determine the effect of each input variable on the driving outcome. That is, for example, the input variables may be scored (e.g., weighted), such as with a number between 0 and 10), based on their effect on the driving outcome. The first ranking of the input variables may thus be determined based on the internal feature ranking of the random forest classifier (e.g., the scoring and/or weighting of the input variables by the random forest).

At step 408, the method 400 may involve providing a screen display including a graphical representation of the input variables automatically arranged based on the first ranking. As an illustrative example, the diagnostic analysis system 100 and/or the processor 110 may arrange a graphical representation corresponding to an input variable with the greatest impact on (e.g., highest correlation with) a driving outcome first within the screen display and may arrange an icon corresponding to an input variable with the least impact on (e.g., lowest correlation with) the driving outcome last within the screen display. A graphical representation of the first ranking may additionally or alternatively be provided as text, a symbol, an icon, and/or the like in association with the set of input variables on the screen display. Moreover, the screen display may be provided on a display, such as the display 112. Further, the screen display may be provided in a relatively short time (e.g., in real-time), such as on the order of milliseconds, following the selection of the driving outcome. To that end, the classification model and/or the diagnostic analysis system 100 may be implemented as responsive to user inputs.

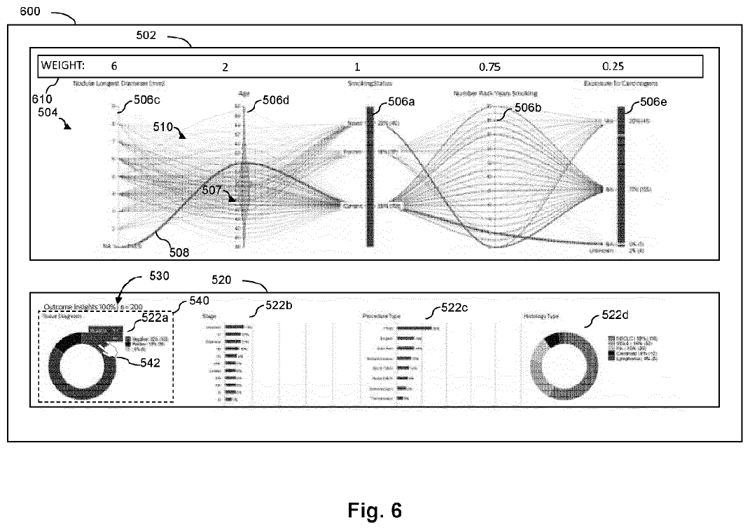

FIG. 6 is a diagrammatic view of screen display 600 (e.g., a GUI) that may be provided at step 408 of the method 400. In particular, FIG. 6 provides an exemplary screen display 600 for the graphical representation of input variables automatically arranged based on a ranking determined based on the driving outcome of tissue diagnosis (522a). In comparison with the screen display 500 of FIG. 5, the order of appearance of the input variables 506a-e within the input window 502 is modified to reflect the respective correlation between the input variables 506a-e and the driving outcome of tissue diagnosis (first outcome 522a). For instance, the input variable 506c (nodule longest diameter (mm)) and the input variable 506d (age) are repositioned from the third and fourth position from the left to the first and second position from the left, respectively. As a result of the modification to the order of appearance of the input variables 506a-e (e.g., in comparison with FIG. 5), the shapes of the curves within the parallel plot 504 may also change. For example, the shape of the current patient curve 508 may be modified so that the current patient curve 508 continues to intersect the axes of the input variables 506a-e at values associated with the records (e.g., a patient record) of the current patient. That is, for example, the values of the current patient curve 508 at each input variable 506a-e may remain the same as the order of appearance of the input variables 506a-e is modified as described herein. Similarly, the similar patient curves 510 may be modified so that the similar patient curves 510 continue to intersect the input variables 506a-e at values associated with the respective records (e.g., patient records) of the similar patients.

In FIG. 6, the input variables 506a-e are arranged based on the first ranking determined at step 406 of the method 400, where the input variable having the highest correlation with the driving outcome is arranged first (e.g., the furthest left) and the input variable having the lowest correlation with the driving outcome is arranged last (e.g., the furthest right). In the illustrated example, the input variable 506c (nodule longest diameter (mm)) may be the most highly correlated with the tissue diagnosis (e.g., the driving outcome). In other words, the input variable 506c may be the most relevant predictor of a particular tissue diagnosis. As an illustrative example, a nodule diameter exceeding 4 mm may be correlated with a positive tissue diagnosis, while a nodule diameter less than or equal to 4 mm may be correlated with a negative tissue diagnosis. Moreover, these diagnoses may be relatively unaffected by values of the other input variables (506a-b and d-e).

As further illustrated in FIG. 6, the ranking of the input variables 506a-e may be indicated via text in a label 610. In the illustrated embodiment, the label 610 illustrates the weighting (e.g., feature importance) assigned to each of the input variables 506a-e based on the classification model. For instance, as described above, the weights may be between 0 and 10 or any other suitable range, where 0 represents a low feature importance and 10 represents a relatively high feature importance or vice versa. To that end, the arrangement of the input variables 506a-e corresponds to an ordering of the input variables 506a-e from highest weight to lowest. Moreover, the label 610 provides an indication of the relative difference in importance (e.g., correlation) between each of the input variables 506a-e.

In some embodiments, the ranking of the input variables 506a-e may additionally or alternatively be represented as a percentage, fraction, decimal, or ordering description (e.g., 1^{st}, 2^{nd}, 3^{rd}, etc.) to provide a representation of the correlation between an input variable and the driving outcome. The ranking may be represented as a plot, a graph, a symbol, an icon, a difference in size, style, and/or the like of the input variables, or any other suitable graphical representation. Further, in some embodiments, the ranking of the input variables 506a-e may be specified without rearranging the input variables 506a-e (e.g., the axes of the input variables 506a-e) within the parallel plot 504. For instance, the ranking of the input variables 506a-e may be specified within the label 610 alone.

Returning to FIG. 4, at step 410, the method 400 may involve receiving a filter criterion associated with the input variables. The filter criterion may be received via a user input from a user device (e.g., user device 114). Further, the filter criterion may correspond to a selection of a subset of the clinical records and/or a selection of a subset of the input variables. For instance, a filter criterion specifying a range or value of data corresponding to an input variable may correspond to a selection of the subset of the clinical records. A filter criterion specifying a particular subset of the set of input variables included in the clinical records (e.g., input variables 310) may correspond to the subset of the input variables. In any case, a filtered dataset of data within the clinical records may be determined based on the filter criterion. As an illustrative example, a user may filter the clinical records so that only the clinical records associated with patients aged between 25 and 35 years are included in the filtered dataset. In such cases, the filtered dataset may include data for each of the input variables in the clinical records but may only include patient records for the subset of patients satisfying the age range. In another example, a user may filter the clinical records so that only data corresponding to the input variables of patient gender and a symptom, such as a cough, are included in the filtered dataset. In such cases, the filtered dataset may include information from the clinical records for each patient included in the clinical records, but this information may only be associated with the input variables of patient gender and symptom. In a further example, a user may filter the clinical records based on a subset of input variables and a value or range of values of an input variable of the subset of input variables.

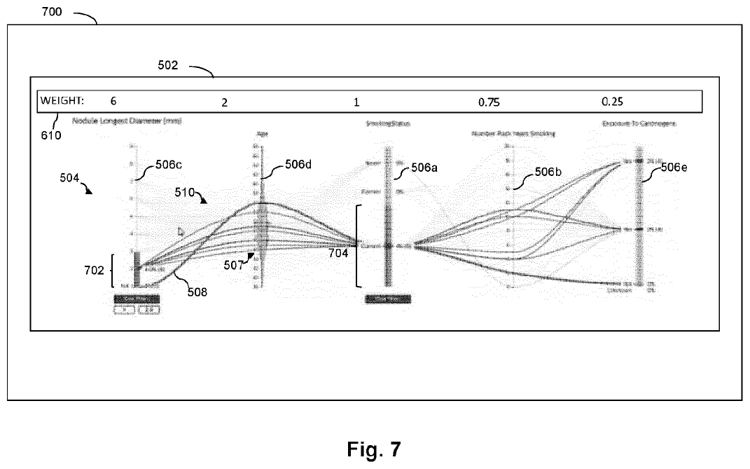

FIG. 7 is a diagrammatic view of a screen display 700 (e.g., a GUI) configured to receive selection of a filter criterion and display the filter criterion. For instance, the screen display includes a first filter criterion 702 associated with the input variable 506c (nodule longest diameter (mm)) and a second filter criterion 704 associated with the input variable 506a (smoking status). In particular, the first filter criterion 702 may correspond to a selection of a subset of clinical records associated with a value of the input variable 506c (nodule longest diameter (mm)) between 1 and 3 mm. To that end, the first filter criterion 702 may correspond to a filtering based on a quantitative input variable. The second filter criterion 704 may correspond to a selection of a subset of clinical records associated with a value of the input variable 506a (smoking status) that is “Current.” To that end, the second filter criterion 704 may correspond to a filtering based on a qualitative input variable. Moreover, the first filter criterion 702 and the second filter criterion 704 may be applied to the clinical records so that the resulting filtered dataset satisfies both the first filter criterion 702 and the second filter criterion 704. To that end, a subset of the curves included in the parallel plot 504 may be displayed based on the filter criteria (e.g., 702 and 704). For instance, the parallel plot 504 may be updated to display similar patient curves 510 corresponding to clinical records that satisfy the filter criteria and to hide (e.g., remove from display) similar patient curves 510 corresponding to clinical records that fail to satisfy the filter criteria. The parallel plot 504 may be similarly updated to display or hide the current patient curve 508 based on whether the clinical records associated with the current patient curve 508 satisfy the filter criteria.

In some embodiments, the filter criteria (702, 704) may be received and subsequently applied simultaneously to generate the filtered dataset. In some embodiments, filter criteria may be applied in a stepwise fashion. As an illustrative example, the first filter criterion 702 may be received and a first filtered dataset may be determined based on the first filter criterion 702. Subsequently, the second filter criterion 704 may be received and applied to the first filtered dataset to determine a second filtered dataset.

While the illustrated filter criteria (702, 704) correspond to selections of a subset of clinical records, a filter criterion may additionally or alternatively correspond to a selection of a subset of input variables, as described above. For instance, while six input variables 506a-e are illustrated in the screen display 700, additional input variables included in a clinical may be added and/or one or more of the input variables 506a-e may be removed from a resulting filtered dataset.

As described above, the filter criterion may be received via a user input. In particular, the user input may correspond to a text entry (e.g., typing), clicking, highlighting, outlining, selecting (e.g., from a list), and/or the like at the screen display 700. That is, for example, a user may interact with the screen display to provide the filter criterion.

With reference now to FIG. 4, at step 412, the method 400 may involve determining a second ranking of the input variables. In particular, the second ranking of input variables may be determined based on the driving outcome, the filter criterion, and the classification model (e.g., classification model 118). For instance, based on the filter criterion, a filtered dataset may be identified, as described above. The second ranking may then be determined based on the filtered dataset and the classification model. More specifically, using the classification model, a correlation between each input variable of the set of input variables associated with the filtered dataset and the data corresponding to the driving outcome associated with the filtered dataset may be determined to determine the second ranking, as generally described above with respect to the first ranking and step 406. Because the filtered dataset may include a subset of the data (e.g., the data corresponding to input variables and/or outcomes) included in the clinical records, the filtered dataset may be different from the clinical records. To that end, the random forest (e.g., classification model 118) may be retrained based on the filtered dataset, which may result in a new feature ranking. In this way, correlations between data corresponding to an input variable and an outcome (e.g., a driving outcome) may also vary between the filtered dataset and the clinical records. Thus, the second ranking may differ from the first ranking.

At step 414, the method 400 may involve modifying the screen display to present the graphical representation of the input variables automatically arranged based on the second ranking. As described above, the second ranking may differ from the first ranking. Accordingly, presenting the graphical representation of the input variables arranged based on the second ranking may involve rearranging the input variables and/or updating an indication of the respective rank of each of the input variables. For instance, providing the screen display of the graphical representation of the input variables arranged based on the first ranking (e.g., at step 408) may involve a first arrangement of icons representative of the input variables, while providing the screen display of the graphical representation of the input variables arranged based on the second ranking (e.g., at step 414) may involve a different, second arrangement of the icons. Further, the screen display may be modified in a relatively short time (e.g., in real-time), following the selection of the filter criterion. To that end, the classification model and/or the diagnostic analysis system 100 may be implemented as responsive to user inputs, as described above.

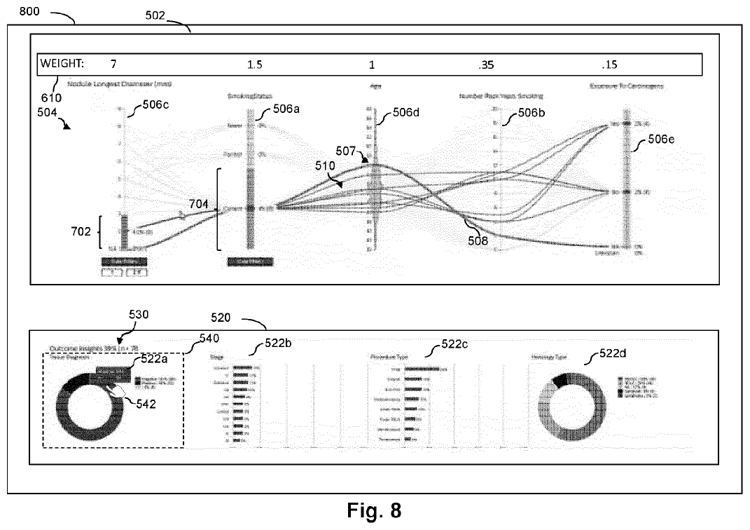

FIG. 8 is a diagrammatic view of an exemplary screen display 800 (e.g., a GUI) for the graphical representation of input variables automatically arranged based on a second ranking. In particular, the screen display 800 illustrates graphical representation of input variables automatically arranged based on a ranking determined based on the classification model, the filter criteria (702, 704), and the driving outcome of tissue diagnosis (e.g., outcome 522a). In comparison with the screen display 600 of FIG. 6 and/or the screen display 700 of FIG. 7, the order of appearance of the input variables 506a-e within the input window 502 is modified to reflect the second ranking (e.g., determined at step 412 of FIG. 4). For instance, the input variable 506d (age) and the input variable 506a (smoking status) are repositioned from the second and third position from the left to the third and second position from the left, respectively. As discussed above with reference to FIG. 6, the modification to the order of appearance of the input variables 506a-e may result in modification of the shapes of the curves within the parallel plot 504. For example, the shape of the current patient curve 508 and/or the shape of the similar patient curves 510 may be adjusted based on the arrangement of the input variables 506a-e.

FIG. 8 further illustrates that, in some embodiments, the graphical representation of the set of outcomes (522a-d) may be modified based on the filtered dataset (e.g., in response to selection of a filter criterion). To that end, because the filtered dataset may include a subset of data corresponding to outcomes in comparison with the data included in all the clinical records, the graphical representation of the outcomes may be modified to reflect the filtered dataset. For instance, the sample size 530 included within the outcome window 520 may be updated based on the number of samples included in the filtered dataset. In the illustrated example, the filtered dataset includes a sample size of 78 patients (e.g., 39% of the total patients included in the clinical records) indicated by the graphical representation of the sample size 530, while the sample size illustrated in FIG. 5 is shown as 200 patients (e.g., 100% of the total patients included in the clinical records) by the graphical representation of the sample size 530. Further, the graphical representations of each of the outcomes 522a-d may be updated to reflect the data associated with this reduced sample size (e.g., with the filtered data set).

Returning to FIG. 4, at step 416, the method 400 may involve identifying a treatment based on the screen display. The treatment (e.g., treatment plan) may correspond to the administration of a medicine, performance of a procedure, such as a surgical procedure, prescription of a regimen, such as physical therapy, a diet, and/or the like, obtaining additional patient data (e.g., running an additional test, obtaining a scan or image of the patient and/or the like), performing no action, and/or the like.

In some embodiments, the treatment may be determined based on the second ranking and/or the driving outcome. For instance, in some embodiments, the driving outcome may correspond to a treatment, such as one or more of the above-mentioned treatments. As an illustrative example, the driving outcome may correspond to a tissue biopsy. For instance, the driving outcome may represent patients that were successfully treated with a biopsy and patients that were successfully treated without a biopsy. Accordingly, because the screen display (e.g., screen display 800) includes a graphical representation of the input variables arranged based on the second ranking, a relationship between each of the input variables and the driving outcome (e.g., a treatment option) may be readily apparent. To that end, comparing the current patient (e.g., the current patient curve 508) with one or more of the similar patient curves with respect to the input variable with the most weight (e.g., a ranking indicative of the highest correlation with the driving outcome) may provide a reliable predictor of an outcome for the current patient. This comparison may be made by visual inspection of the parallel plot 504, for example, and/or by providing filter criterion that narrow the dataset of clinical records to patients similar to the current patient with respect to the input variables that are the strongest predictors of the driving outcome. Continuing with the above example, if the driving outcome is tissue biopsy and the input variable ranked as the strongest predictor of the driving outcome is patient age, a clinician may determine whether to perform a tissue biopsy on a current patient based on whether tissue biopsy was a successful treatment for one or more patients whose age is similar to (e.g., within a threshold range with respect to) the age of the current patient. For instance, the clinician may filter the clinical records based on an age range including the age of the current patient and may determine whether or not to perform a tissue biopsy on the current patient based on the tissue biopsy results of other patients within the age range.

Additionally or alternatively, the diagnostic analysis system 100 may identify the treatment. For instance, in response to an input variable having a correlation with a driving outcome that satisfies a threshold, the diagnostic analysis system 100 may compare the data of the current patient to the data of other patients for the input variable. For instance, the diagnostic analysis system 100 may determine a correlation between the data of the current patient for the input variable and the data of one or more of the other patients for the input variable. In some embodiments, for example, the diagnostic analysis system 100 may determine this correlation using a classification model (e.g., the classification model 118). As an illustrative example, if the patient is determined to be strongly correlated (e.g., showing a correlation satisfying a threshold) with a set of patients based on the input variable, the diagnostic analysis system 100 may identify the treatment as a treatment associated with one or more of the set of patients, such as a majority of the set of patients. Further, if the patient is determined to not be strongly correlated (e.g., showing a correlation failing a threshold) with the set of patients, the diagnostic analysis system 100 may determine the treatment to be opposite the treatment associated with the one or more of the set of patients or may determine the treatment to be a follow-up test, procedure, and/or the like to acquire more information. The diagnostic analysis system 100 may additionally or alternatively filter the clinical records based on data of the current patient for the input variable and may determine a treatment for the current patient based on the treatment results of other patients within the filter.

Further, in some embodiments, a graphical representation of the identified treatment may be provided on a screen display, such as screen display 800. For instance, text, a numeral, symbol, icon, image, and/or the like may be provided on the screen display to indicate the identified treatment.

While the identification of a treatment is illustrated and described above with respect to the second ranking of the input variables, embodiments are not limited thereto. To that end, the treatment may additionally or alternatively be identified based on the first ranking of the input variables (e.g., a ranking of the input variables without filter criterion) or any other suitable ranking (e.g., based on any filtered dataset of the clinical records). Moreover, the identification treatment may be determined based on one or more outcomes, such as a driving outcome and another outcome and/or multiple driving outcomes. In some embodiments, for example, a set of driving outcomes may be selected (e.g., at step 404), and rankings of the input variables may be determined based on the set of driving outcomes. In any case, by determining the relationship between input variables and one or more driving outcomes, more relevant patients may be identified as similar to a current patient, and based on these more relevant patients, a suitable treatment plan for the current patient may reliably be identified.

At step 418, the method 400 may include performing the treatment identified at step 416. In some embodiments, a clinician may perform the treatment or a portion thereof. For instance, the clinician may administer a medicine, perform a procedure, such as a surgical procedure, prescribe a regimen to a patient and/or the like. Additionally or alternatively, the diagnostic analysis system 100 may perform a portion of the treatment. For instance, the diagnostic analysis system 100 may automatically schedule an appointment, generate and/or transmit (e.g., to a patient and/or a pharmacy) a prescription, submit an order for a test, and/or the like.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a data store comprising clinical records associated with a plurality of patients, wherein, for each of the plurality of patients, the clinical records comprise data corresponding to a set of inputs associated with a medical condition of the patient and data corresponding to a set of outcomes associated with the medical condition of the patient; and
a processor circuit in communication with the data store and a user input device, wherein the processor circuit is configured to:
obtain the clinical records via the data store;
receive, via the user input device, a selection of a driving outcome from among the set of outcomes;
determine a first ranking of the set of inputs based on the driving outcome and a classification model; and
provide, at a display in communication with the processor circuit, a screen display including a graphical representation of the set of inputs automatically arranged based on the first ranking.

2. The system of claim 1, wherein the processor circuit is configured to, in response to receiving a selection of a filter criterion associated with the set of inputs via the user input device:
determine a second ranking of the set of inputs based on the driving outcome, the filter criterion, and the classification model; and
modify the screen display provided on the display to present the graphical representation of the set of inputs automatically arranged based on the second ranking.

3. The system of claim 2, wherein the filter criterion corresponds to at least one of:
a selection of a subset of the clinical records based on data corresponding to an input of the set of inputs, or
a selection of a subset of the set of inputs.

4. The system of claim 2, wherein the processor circuit is configured to determine the second ranking further based on:
identifying, based on the filter criterion, a filtered dataset comprising a subset of the data corresponding to the set of inputs and the data corresponding to the set of outcomes; and
identifying, based on the classification model and the filtered dataset, a respective correlation between each input of the set of inputs associated with the filtered dataset and the data corresponding to the driving outcome associated with the filtered dataset.

5. The system of claim 2, wherein the screen display comprises a set of icons, wherein each of the set of icons correspond to a respective input of the set of inputs.

6. The system of claim 5, wherein the processor circuit is configured to provide the graphical representation of the set of inputs automatically arranged based on the first ranking based on a first arrangement of the set of icons, and
wherein the processor circuit is configured to present the graphical representation of the set of inputs automatically arranged based on the second ranking based on a second arrangement of the set of icons.

7. The system of claim 1, wherein the screen display comprises a graphical representation of the set of outcomes.

8. The system of claim 7, wherein the processor circuit is further configured to:
output, to the screen display, the graphical representation of the set of outcomes based on the data corresponding to the set of outcomes; and
in response to receiving a selection of a filter criterion associated with the set of inputs via the user input device:
identify a filtered dataset, wherein the filtered dataset comprises a subset of the data corresponding to the set of outcomes; and
modify the graphical representation of the set of outcomes based on the subset of the data corresponding to the set of outcomes.

9. The system of claim 1, wherein the processor circuit is configured to determine the first ranking further based on:
identifying, based on the classification model, a respective correlation between the data corresponding to each input of the set of inputs and the data corresponding to the driving outcome.

10. The system of claim 1, wherein the classification model comprises at least one of a random forest, a logistic regression model, or a cox regression model.

11. The system of claim 1, wherein the set of inputs comprise at least one of an age, gender, race, symptom, clinical test result, family history, or diagnosis.

12. The system of claim 1, wherein the set of outcomes comprise at least one of a treatment, a mortality rate, a morbidity rate, a level of severity, or a diagnosis confidence metric associated with the medical condition.

13. The system of claim 1, wherein the processor circuit is further configured to:
provide, at the display, a graphical representation of the data corresponding to the set of inputs based on the graphical representation of the set of inputs.

14. The system of claim 13, wherein the processor circuit is further configured to:
in response to receiving a selection of a filter criterion associated with the set of inputs via the user input device:
identify a filtered dataset, wherein the filtered dataset comprises a subset of the clinical records based on data corresponding to an input of the set of inputs; and
modify the graphical representation of the set of inputs based on the subset of the clinical records.

15. The system of claim 13, wherein the graphical representation of the data corresponding to the set of inputs comprises a histogram.

16. A method, comprising:
obtaining, by a processor circuit, clinical records associated with a plurality of patients via a data store, wherein, for each of the plurality of patients, the clinical records comprise data corresponding to a set of inputs associated with a medical condition of the patient and data corresponding to a set of outcomes associated with the medical condition;
receiving, at the processor circuit, a selection of a driving outcome from among the set of outcomes via a user input device;
determining, by the processor circuit, a first ranking of the set of inputs based on the driving outcome and a classification model; and
providing, by the processor circuit, a screen display including a graphical representation of the set of inputs automatically arranged based on the first ranking on a display in communication with the processor circuit.

17. The method of claim 16, comprising:
in response to receiving, at the processor circuit, a selection of a filter criterion associated with the set of inputs via the user input device:
determining, by the processor circuit, a second ranking of the set of inputs based on the driving outcome, the filter criterion, and the classification model; and
modifying, by the processor circuit, the screen display provided on the display to present the graphical representation of the set of inputs automatically arranged based on the second ranking.

18. The method of claim 16, comprising:
identifying a treatment for a patient of the plurality of patients based on the graphical representation of the set of inputs arranged based on the first ranking; and
performing the treatment.
